(19) United States
(12) Patent Application Publication
(10) Pub. No.: US 2026/0000762 A1
(43) Pub. Date: Jan. 1, 2026

(54) CONTROLLED EXPRESSION OF A TRANSGENE IN HUMAN T OR NK CELLS FOR USE IN CELLULAR IMMUNOTHERAPY

(71) Applicants: NUTRITHERAGENE, CLERMONT FERRAND (FR); INSTITUT NATIONAL DE RECHERCHE POUR L'AGRICULTURE, L'ALIMENTATION ET L'ENVIRONNEMENT, PARIS (FR); UNIVERSITÉ CLERMONT AUVERGNE, CLERMONT FERRAND (FR); CENTRE HOSPITALIER UNIVERSITAIRE DE CLERMONT-FD, CLERMONT FERRAND CEDEX (FR)

(72) Inventors: Alain BRUHAT, CHANONAT (FR); Aurore DOUGÉ, MEZEL (FR); Paul ROUZAIRE, CHANONAT (FR)

(21) Appl. No.: 18/880,788

(22) PCT Filed: Jul. 5, 2023
(86) PCT No.: PCT/EP2023/068496

§ 371 Date: Jan. 2, 2025

(30) Foreign Application Priority Data
Jul. 5, 2022 (EP) 22306008.8

(51) Int. Cl. A61K 40/31; A61K 38/20; A61K 40/11

(52) U.S. Cl. CPC A61K 40/31; A61K 38/2066; A61K 40/11; A61K 2239/23

(57) ABSTRACT

The present invention concerns a human T cell or NK cell comprising a nucleic acid construct which comprises a transgene which is placed under the control of a regulatory polynucleotide inducible by a deficiency in at least one essential amino acid. and cellular immunotherapy employing said human T cell or NK cell.

The present invention concerns a human T cell or NK cell comprising a nucleic acid construct which comprises a transgene which is placed under the control of a regulatory polynucleotide inducible by a deficiency in at least one essential amino acid, and cellular immunotherapy employing said human T cell or NK cell.

Cellular immunotherapy is currently expanding. For instance, in oncology, this therapeutic approach is using immune cells to rebalance the impaired anti-tumor immunosurveillance. This principle is widely used in the treatment of hematological malignancies, with old cell therapies such as allogeneic hematopoietic stem cell transplantation (allo-HSCT). Allo-HSCT basically involves replacing a patient's diseased bone marrow with healthy bone marrow collected from an HLA-matched donor. It represents a powerful cell therapy strategy, allowing bone marrow and immune system reconstitution but also anti-tumor effect of donor lymphocytes (Graft-versus-Leukemia or GvL effect). Unfortunately, this procedure also exhibits high toxicity such as graft versus host disease (GvHD) and infections, which limit the indications for curative strategies. These complications represent a major cause of morbidity and mortality, poor quality of life and an additional economic cost, due in particular to the expensive therapies to treat them and the increased hospitalization time for patients.

More recently, CAR-T (Chimeric Antigen Receptor-T) cells therapy represents a new approach for cancer treatment. CAR-T cells are autologous T cells collected from the patient and modified ex vivo to express a chimeric antigen receptor (CAR) specific for a tumor antigen of interest. The CAR is composed by (i) an antigen-binding domains (a single-chain variable fragment scFv derived from an antibody), (ii) signaling domains of the T-cell receptor (TCR), and (iii) additional costimulatory domains. These lymphocytes are then reinjected into the patient, and will be able to self-activate upon recognition of their target antigen, and directly kill the tumor cells carrying this antigen. For example, anti-CD19 CAR-T cells have obtained Marketing Authorization for several years in the treatment of certain haematological malignancies in relapse or refractory to conventional chemotherapy treatments, and the global development of CAR-T cells is booming, including for the treatment of solid tumors. However, this innovative therapeutic strategy has points for improvement, particularly in terms of efficacy since a significant percentage of patients will see their CAR-T cells become less competent over time following, for example, a CAR exhaustion phenomenon. Additionally, where CAR-T cell toxicity develops (e.g. with onset of symptoms of cytokine release syndrome or central nervous system toxicity), it may be needed to curb activity of CAR-T cells in a reversible manner, for example by making the CAR-T cell “exhausted” so that it is reversibly braked rather than irreversibly destroyed.

Another recent major cellular therapy strategy is TCR transgenic T cells. Similarly to CAR-T cells, TCR transgenic T cells are modified to express a receptor specific for a tumor antigen, but this time, the TCR looks like endogen TCR and recognizes tumor antigen presented by Major Histocompatibility Complex (MHC), allowing TCR recognition of more antigens than CAR-T cells since CAR-T recognize only surface antigens. This strategy is particularly expanding in the treatment of solid tumors.

Still another recent antitumoral approach consists in the use of primary NK cells or modified CAR-NK cells infusion, with promising results in the field of hematological malignancies or solid tumors. This last option needs a careful tuning to avoid issues highlighted in CAR-T cells.

Cellular immunotherapy consists also in promising approaches in all pathologies where T cells are involved, and numerous trials are ongoing in auto-immune or allergic diseases or graft rejection using either CAR-T cells or TCR transgenic T cells.

However, to date there are still many difficulties in controlling these cells once reinjected into the patient, either to increase their therapeutic action or to limit their toxicity. It therefore appears important to be able to provide a flexible and easily reversible gene expression regulation system to control the effects of these cells as simply as possible.

The present invention highlights a new use of a gene expression regulatory technology disclosed in the international patent application published as WO 2013/068096, herein called NUTRIREG. In mammals, essential amino acids (EAAs) are not synthesized by the body and must therefore be provided by food. In case of lack, the organism must absolutely adapt, which is why it activates a specific signaling pathway, the GCN2-ATF4 pathway, leading to the overexpression of a transcription factor (ATF4) present in all the cells of the organism. This transcription factor will then bind to the DNA at the level of the promoter of specific target genes (Amino Acid Responsive—AARE sequences) and activate their transcription. These genes play a key role in the adaptation process to nutritional stress. This GCN2-ATF4 signaling pathway was therefore used to develop a system for regulating the expression of a transgene. The NUTRIREG system is based on the association (i) of an artificial promoter (based on AARE sequences) strongly inducible by a deficiency in one EAA which controls the expression of a transgene (AARE-Gene) with (ii) a food deficient in an EAA which leads to a sharp drop in the blood concentration of the limiting EAA and allows the induction of the GCN2-ATF4 signaling pathway. After having delivered the AARE-Gene plasmid to the target tissue using a viral vector, the expression of the transgene can thus be induced after the consumption of a diet deficient in one EAA (FIG. 1). Interestingly, this pathway can be quickly turned-off via the consumption of the missing EAA, which offers an easily reversible regulation system, and devoid of adverse effects since it involves a physiological nutritional pathway. Proof of the functionality of this system has already been provided in mice, in different organs (liver, pancreas, brain, eye), but also in glioblastoma tumor cells thanks to the use of a pro-apoptotic gene (Chaveroux et al, Nat Biotechnol, 2016, 34, 746-751).

The NUTRIREG technology is used in this invention to control the cDNA expression of a protein having a therapeutic application in cellular immunotherapy. This construction, inserted in a viral vector, can be directly integrated into donor T or NK cells, or into modified T or NK cells, before reinjection into the patient. Using an essential amino acid-deficient mixture will allow expression of the peptide drug on demand when needed. The use of NUTRIREG in cellular immunotherapy opens up numerous preventive or curative therapeutic possibilities, depending on the drug gene expressed, and the time at which it is expressed, whether to curb toxicity or, on the contrary, to stimulate the efficacy of the cell therapy studied.

### SUMMARY OF THE INVENTION

The invention relates to a human T cell or NK cell comprising a nucleic acid construct which comprises:
i) a regulatory polynucleotide comprising a minimal promoter and at least one AARE (amino acid response element) nucleic acid sequence, said regulatory polynucleotide being activated in said T or NK cell when said T or NK cells is activated, and upon consumption of a diet deficient in at least one essential amino acid; and
ii) a transgene which is placed under the control of the said regulatory polynucleotide.

The invention further relates to said human T cell or NK cell for use for cellular immunotherapy.

A method for preparing a human T cell or NK cell according to the invention is also provided, wherein a human T or NK cell is transfected or transduced with a vector comprising a nucleic acid construct which comprises:
i) a regulatory polynucleotide comprising a minimal promoter and at least one AARE (amino acid response element) nucleic acid sequence, said regulatory polynucleotide being activated in said T or NK cell when said T or NK cell is activated, and upon exposure to a deficiency in at least one essential amino acid; and
ii) a transgene which is placed under the control of the said regulatory polynucleotide.

### DETAILED DESCRIPTION

The GCN2-ATF4 signaling pathway is ubiquitous in cells but, depending on the tissue or organ, the pathway may not be activated by the nutritional modulation system. For instance, EAA deprivation cannot induce activation of the GCN2-ATF4 pathway in muscles in mice. The inventors demonstrated that the GCN2-ATF4 pathway is inducible by an EAA starvation in human T cells, but initial in vitro experiments failed as the inventors also found that activation of the human T lymphocyte is essential for the activation of GCN2 kinase. Thus the expression of the transgene is only possible when the T cells are activated and the EAA deprivation induces activation of the GCN2-ATF4 pathway. These results are expected to be transposable to human NK cells, since a NK cell is basically a cytotoxic T cell without a TCR.

### NUTRIREG T-cells or NK-cells

The invention relates to a human T-cell or NK cell that comprises a nucleic acid construct, which comprises:
i) a regulatory polynucleotide comprising a minimal promoter and at least one AARE (amino acid response element) nucleic acid sequence, said regulatory polynucleotide being activated in a subject upon consumption of a diet deficient in at least one essential amino acid; and
ii) a transgene which is placed under the control of the said regulatory polynucleotide.

The invention also relates to a human T cell or NK cell comprising a nucleic acid construct which comprises:
i) a regulatory polynucleotide comprising a minimal promoter and at least one AARE (amino acid response element) nucleic acid sequence, said regulatory polynucleotide being activated in said T or NK cell when said T or NK cell is activated, and upon exposure to a deficiency in at least one essential amino acid; and
ii) a transgene which is placed under the control of the said regulatory polynucleotide.

### Human T Cells or NK Cells, and Transgenes

According to some embodiments, the human T-cell of the invention is a chimeric antigen receptor T (CAR-T) cell (which includes CAR-Tregs), or a T cell receptor (TCR) transgenic T cell.

In these embodiments, the transgene is selected so as to stimulate efficacy of cellular immunotherapy with said CAR-T cell or transgenic TCR T cell, or to curb toxicity induced by said CAR-T cell, or transgenic TCR T cell. In particular, the transgene is a transgene that prevents CAR-T cell exhaustion.

Suitable transgenes that stimulate efficacy of cellular immunotherapy with CAR-T cell or transgenic TCR T cell include, according to recent promising studies, c-JUN, T-bet, IL-7, IL-12, IL-15, IL-18, IL-21, IL-23 (to reverse or delay T-cell exhaustion or to stimulate those modified T cells) (Poorebrahim, et al., Oncogene 40.2 (2021): 421-435; Pietrobon et al., International Journal of Molecular Sciences 22.19 (2021): 10828).

Suitable transgenes that curb toxicity induced by CAR-T cell or transgenic TCR T cell include inhibitory receptors (such as PD-1, CTLA-4, TIM-3, LAG-3), or immunosuppressive factors (such as IL-35, IL-10, TGF-β, FoxP3, IDO, TOX, Eomes) (Poorebrahim, et al., Oncogene 40.2 (2021): 421-435; Pietrobon et al., International Journal of Molecular Sciences 22.19 (2021): 10828).

These human T-cells according to the invention are in particular intended for the treatment of cancers, such as hematological malignancies or solid cancers.

According to some embodiments, the human T-cell of the invention is intended for allogeneic transplantation, or for treating a hematologic cancer.

In a first aspect of these embodiments, the transgene is selected so as to promote human T cell differentiation into regulatory T cells (Treg), or in type 1 regulatory T (Tr1) cells.

Suitable transgenes that promote human T cell differentiation into (i) Tr1 include a transgene encoding a cytokine such as IL-10, TGF-β, IFN-α, or IL-6 (Roncarolo et al., Immune-Mediated Diseases, Immunity, Volume 49, Issue 6, 2018), or (ii) Treg include mainly FoxP3. Preferably, the transgene that promotes human T cell differentiation into Tr1 is a transgene encoding IL-10.

According to this aspect, expression of the transgene is to be induced when signs of GvHD are detected, in order to inhibit or control GvHD.

According to a second aspect of these embodiments, the human T-cell which is intended for use for treatment of a hematologic cancer by allogeneic transplantation comprises a transgene that stimulates efficacy of cellular immunotherapy with an individual, i.e. a human or a non-human mammal. For instance, the transgene encodes a stimulatory cytokine such as IL-2, IFN-γ, TNF-α, IL-7, IL-15 (Ringdén, et al., British journal of haematology 147.5 (2009): 614-633).

According to some embodiments, the human T-cell of the invention is intended for controlling allograft rejection of solid organ transplant, or for use for treating an autoimmune disease. In these embodiments, the human T-cell is a human CAR-Treg, more particularly an antigen-specific CAR-Treg, wherein the antigen is a transplant-specific alloantigen, or an auto-antigen associated with the auto-immune disease.

Antigen-specific human CAR-Tregs can be generated by isolating polyclonal Tregs (from peripheral blood of an individual) and transducing them with an antigen-specific CAR construct, or by cotransducing CD4⁺ or CD3⁺ T cells with antigen-specific CAR constructs and FoxP3 cDNA (Arjomandnejad et al. Biomedicines 2022, 10 (2), 287).

In these embodiments, the transgene is selected so as to stimulate CAR-Treg efficacy and/or prevent CAR-Treg exhaustion, and includes c-JUN, T-bet, IL-7, IL-12, IL-15, IL-18, IL-21, IL-23, as already mentioned above.

According to some embodiments, the human cell that comprises the nucleic acid construct, including the regulatory polynucleotide and transgene, is a human NK cell. As used herein a the term NK cell includes NK cells and modified NK cells, such as CAR-NK cell.

In these embodiments, the transgene is selected so as to stimulate efficacy of cellular immunotherapy with said human NK cell or CAR-NK cell of the invention. In particular, the transgene is a transgene that enhances activation or proliferation of NK cells, or CAR-NK cells.

Suitable transgenes include NKG2D, IL-12, IL-15, or IL-18 (Shimasaki N, et al., Nat Rev Drug Discov. 2020 March; 19 (3):200-218; Daher M. et al., Blood. 2021 Feb. 4; 137 (5):624-636; Wang X. et al., Blood Adv. 2020 May 12; 4 (9):1950-1964.).

These human NK cells or CAR-NK cells according to the invention are in particular intended for the treatment of hematological malignancies or solid tumors.

### Nucleic Acid Construct and Regulatory Polynucleotide

The nucleic acid construct comprises:
i) a regulatory polynucleotide comprising a minimal promoter and at least one AARE (amino acid response element) nucleic acid sequence, said regulatory polynucleotide being activable or activated in a subject upon consumption of a diet deficient in at least one essential amino acid; and
ii) a transgene which is placed under the control of the said regulatory polynucleotide.

Alternatively, the nucleic acid construct comprises:
i) a regulatory polynucleotide comprising a minimal promoter and at least one AARE (amino acid response element) nucleic acid sequence, said regulatory polynucleotide being activated in a T or NK cell when said T or NK cell is activated, and upon exposure to a deficiency in at least one essential amino acid; and
ii) a transgene which is placed under the control of the said regulatory polynucleotide.

Such a nucleic acid construct has been described in the international patent applications published as WO 2013/068096 A1 and WO2017/207744 A1 that are herein incorporated in their entirety.

The regulatory polynucleotide comprises a minimal promoter and at least one AARE (amino acid response element) nucleic acid sequence.

As used herein, a “minimal promoter” is intended to mean a promoter including all the required elements to properly initiate transcription of a gene of interest positioned downstream. The expressions “minimal promoter” and “core promoter” are considered as equivalent expressions. A skilled in the art understands that the “minimal promoter” includes at least a transcription start site, a binding site for a RNA polymerase and a binding site for general transcription factors (TATA box).

Suitable minimal promoters are known for a skilled artisan.

In some embodiments, a minimal promoter is selected from the group consisting of the promoter of the thymidine kinase (TK), the promoter of the β-globin, the promoter for cytomegalovirus (CMV), the SV40 promoter and the like.

As used herein, an “AARE” or “amino acid response element” denotes a nucleic acid sequence which is bound by the activating transcription factor 4 (ATF4), after activation of the GCN2-eIF2α-ATF4 pathway by deficiency in an essential amino acid (EAA), and thereby induce expression of a target gene driven by the AARE.

In mammals, after consumption of a diet deficient in one EAA, the blood concentration of the limiting EAA decreases rapidly and greatly, triggering an ubiquitous adaptive process referred to as the amino-acid response pathway. The initial step of this pathway is the activation of the mammalian GCN2 protein kinase by uncharged tRNAs. GCN2 then phosphorylates the a subunit of eukaryotic initiation factor 2 (eIF2α) on serine 51, leading to upregulation of the translation of the ATF4. Once induced, ATF4 activates transcription of specific target genes through binding to the AARE. The GCN2-eIF2α-ATF4 pathway can be rapidly turned off by the administration of the missing EAA.

This pathway can also be observed at the cellular level. Indeed the amino-acid response pathway is also triggered in activated T or NK cells exposed to a deficiency in at least one EAA, for instance when activated T or NK cells are cultivated in a medium deficient in at least one EAA.

According to some embodiments, the amino acid response element (AARE) nucleic acid sequence is selected in the group consisting of the nucleic acid sequences SEQ ID NO: 1, SEQ ID NO: 2, SEQ ID NO: 3, SEQ ID NO: 4 and SEQ ID NO: 5.

The regulatory polynucleotide which comprises at least one AARE may include at least 2, at least 3, at least 4 or at least 5 AARE nucleic acid sequences. The expression “at least one AARE nucleic acid sequence” thus includes e.g. 1, 2, 3, 4, 5, 6, 7, 8, 9, 10, 11, 12, 13, 14, 15, 16, 17, 18, 19, or 20 AARE nucleic acid sequences.

In certain embodiments, the regulatory polynucleotide comprises at least two AARE nucleic acid sequences. In some other embodiments, the regulatory polynucleotide comprises from 1 to 20 AARE nucleic acid sequences, preferably from 1 to 10 AARE nucleic acid sequences. In certain embodiments, the regulatory polynucleotide comprises from 2 to 6 AARE nucleic acid sequences.

In some embodiments, the regulatory polynucleotide comprises 2 AARE nucleic acid sequences selected from the group consisting of the nucleic acid sequences SEQ ID NO: 2 and SEQ ID NO: 4. In some embodiments, the regulatory polynucleotide comprises 6 AARE nucleic acid sequences of sequence SEQ ID NO: 1.

In certain embodiments, the at least two AARE nucleic acid sequences may be identical or distinct.

According to an embodiment, the regulatory polypeptide comprises the Thymidine kinase (Tk) minimal promoter and six copies of the AARE nucleic acid sequence from the TRIB3 gene, and comprises or consists of a sequence as shown in SEQ ID NO: 6.

The regulatory polynucleotide construct thus comprises at least one AARE nucleic acid sequence placed immediately upstream of the minimal promoter which controls the expression of the transgene placed downstream.

The regulatory polynucleotide is activated in T or NK cells of a subject upon consumption of a diet deficient in at least one essential amino acid.

The subject is a human or a non-human mammal, preferably a human. In some embodiments, the non-human mammal is selected from the group consisting of a mouse, a rat and the like; a primate such as a chimp, a monkey, and the like.

In mammals, nine EAAs must be supplied in the diet, and a lack of any one of them can induce the AARE-driven expression system.

As used herein, an “essential amino acid” includes histidine (His, H), isoleucine (Ile, I), leucine (Leu, L), Lysine (Lys, K), methionine (Met, M), phenylalanine (Phe, F), threonine (Thr, T), tryptophan (Trp, W) and valine (Val, V).

As used herein, a “diet deficient in at least one essential amino acid” is intended to mean a diet deficient in 1, 2, 3, 4, 5, 6, 7, 8 or 9 essential amino acid(s). According to some embodiments, the diet is deficient in Leucine.

The regulatory polynucleotide is activated in an activated T or NK cell when exposed to a deficiency in at least one essential amino acid, for instance when cultivated in a medium deficient in at least one essential amino acid.

The cell is a human cell or a non-human mammal cell, preferably a human cell. In some embodiments, the non-human mammal is selected from the group consisting of a mouse, a rat and the like; a primate such as a chimp, a monkey, and the like.

For the cells, nine EAAs must be supplied in the diet, and a lack of any one of them can induce the AARE-driven expression system.

As used herein, a “medium deficient in at least one essential amino acid” is intended to mean a diet deficient in 1, 2, 3, 4, 5, 6, 7, 8 or 9 essential amino acid(s). According to some embodiments, the diet is deficient in Leucine.

### Activation of the Regulatory Polynucleotide

For activation of the regulatory peptide, a diet deficient in at least one essential amino acid is administered to the subject, i.e. human or non-human mammal as defined above.

According to preferred embodiments, the subject has been starved before the diet deficient in at least one essential amino acid is administered.

For activation of the regulatory peptide in cells in vitro, cells are cultivated in a medium deficient in at least one essential amino acid, preferably for at least 2 h, at least 6 h, at least 10 h, and preferably at least 16 h.

### Cellular Immunotherapy with NUTRIREG T Cells or NK Cells

The human T cell or NK cell according to the invention is for use for cellular immunotherapy.

It is also provided a method of cellular immunotherapy of an subject in need thereof, which comprises administering said subject with a T cell or NK cell according to the invention.

The cellular immunotherapy is intended for an subject that is a human or a non-human mammal, preferably a human. In some embodiments, the non-human mammal is selected from the group consisting of a mouse, a rat and the like; a primate such as a chimp, a monkey, and the like.

In some embodiments, the T cell or NK cell is autologous to the human subject. In some embodiments, the T cell or NK cell is allogenic to the human subject.

The cellular immunotherapy non limitatively includes treating cancer, controlling allograft rejection of solid transplant, or treating an auto-immune disease.

The cancer may be a solid cancer or a hematologic cancer.

At some point(s) in time, the subject who/that is administered with the human T cell or NK cell further receives a diet deficient in at least one essential amino acid to induce transgene expression.

It is an advantage of the human cells according to the invention, especially the human T cells according to the invention, that transgene expression is induced by EAA deprivation (or activation of AARE nucleic acids) only in those human cells, especially T cells, that are activated.

This enables expression of the transgene only where needed, by exposing the human

T cells or NK cells of the invention to EEA deficiency. Indeed, in addition to be induced by an endogenous signaling pathway (GCN2-ATF4) which is food inducible, one of the major advantages of the NUTRIREG-T cell or NK cell system is the possibility of On/Off modulation of the transgene expression, with control of this expression over time.

In an embodiment of the invention, T cells or NK cells of the invention which are administered are not activated. Transgene expression will be only induced in the cells once they are activated in the subject, in particular in case of inflammation, and only when this subject will receive a diet deficient in at least one essential amino acid.

A first application for the human T cells of the invention is in the context of allogeneic stem cell transplantation (allo-SCT) which is the treatment of choice of many haematological malignancies. Allogeneic donor T cells recognize minor histocompatibility antigens (minor H antigens) which results in elimination of leukaemic cells, a phenomenon called the graft versus leukaemia (GvL) effect. Because many minor H antigens are expressed ubiquitously, the GvL effect is often accompanied by the destruction of a patient's normal tissues, the graft versus host disease (GvHD), which is a major side-effect of allo-SCT.

A preventive or curative control of GvHD could be achieved with a human T cell of the invention comprising a transgene promoting the expansion of human T lymphocytes with an anti-inflammatory profile, to slow down the cytotoxic response responsible for GvHD. In this aspect, donor T cells (present in allo-stem cells to be grafted in a subject in need thereof, or in the form of Donor Lymphocytes Injection) are genetically modified ex vivo with the NUTRIREG system including a Treg or Tr1-inducing transgene, before reinjection into the subject. This allows, by simple consumption for a few hours of a diet deficient in an EAA, (i) to express this Treg or Tr1-inducting transgene (usually an anti-inflammatory cytokine) in activated T cells (which are responsible of GvHD), and (ii) to promote the polarization of the T response towards a T regulatory phenotype (Treg or Tr1).

Conversely, in order to regain the graft versus leukemia (GvL) effect in the event of a relapse of the hematologic disease in the subject, one could also consider reboosting the donor T lymphocytes using a stimulatory cytokine transiently expressed under the dependence of EEA deprivation.

Hence, according to some embodiments, the human NUTRIREG T-cell is for use for treating cancer, in particular a hematologic cancer by allogeneic transplantation. The hematologic cancer is in particular leukemia, lymphoma, or multiple myeloma.

According to some embodiments, the human T cell is for use for treatment of a hematologic cancer by allogeneic transplantation, and wherein:
the expression of the transgene promotes human T cell differentiation into Treg or Tr1 and prevents or treats graft versus host disease (GvHD), or
the expression of the transgene stimulates efficacy of cellular immunotherapy with said human T-cell inducing or stimulating graft versus leukemia (GvL) effect.

In some aspects, the transgene promotes human T cell differentiation into regulatory T cells (Treg or Tr1); for instance the transgene encodes a cytokine such as IL-10, TGF-β, IFN-α, and IL-6 or a transcription factor such as FoxP3. Expression of the transgene that promotes human T cell differentiation into Treg or Tr1 prevents or treats graft versus host disease (GvHD). In this aspect, a repeated induction of the transgene that promotes human T cell differentiation into Treg or Tr1 is intended by intermittent consumption of a diet deficient in one EAA.

In the context of controlling the cytotoxic response of GvHD, the On/Off modulation enabled by the NUTRIREG-T cell system is a key asset since the consumption of the missing EAA will stop the expression of the transgene that promotes human T cell differentiation into Treg or Tr1, such as an anti-inflammatory cytokine, in due time. NUTRIREG system is thus highly advantageous because it allows transient expression of the transgene thus preserving anti-tumoral effect of the graft, which is not possible with the immunosuppressive drugs currently used. Another advantage is that the transgene will be expressed only in activated T cells since the GCN2-ATF4 pathway cannot be induced in quiescent T cells, which represents an essential safety control.

In other aspects, the transgene stimulates efficacy of cellular immunotherapy with said human T-cell, to regain GvL effect; for instance the transgene encodes stimulatory cytokines such as IL-2, IFN-γ, TNF-α, IL-7, IL-15. Expression of the transgene that stimulates efficacy of cellular immunotherapy with said human T-cell induces or stimulates GvL effect. In this aspect, a repeated induction of the transgene that stimulates efficacy of cellular immunotherapy with said human T-cell is intended by intermittent consumption of a diet deficient in one EAA, when it is suspected that a relapse of the hematologic disease occurs in the subject.

A second application concerns the therapeutic strategy of human CAR-T cells or TCR transgenic T cells of the invention, either with the aim of limiting their toxicity using a transgene encoding an inhibitory receptor (such as PD-1, CTLA-4, TIM-3, LAG-3), or immunosuppressive factor (such as IL-35, IL-10, TGF-β, FoxP3, IDO, TOX, Eomes), or on the contrary to stimulate their activity. Human CAR-T cells or TCR transgenic T cells according to the invention could allow transient expression of a gene of interest in preventing the exhaustion of the T cells. Encouraging results have been described in the literature regarding c-JUN, which when overexpressed makes the CAR-T cells resistant to exhaustion (Lynn et al., Nature 576, 293-300 (2019)). A transient and repeated expression of c-JUN under the dependence of NUTRIREG in the CAR-T cell TCR transgenic T cell could thus make it resistant to exhaustion and promote the maintenance of its efficacy over time. Some results are also promising with T-bet, IL-7, IL-12, IL-15, IL-18, IL-21, IL-23, to reverse or delay T-cell exhaustion or to stimulate modified T cells (Poorebrahim, et al., Oncogene 40.2 (2021): 421-435; Pietrobon et al., International Journal of Molecular Sciences 22.19 (2021): 10828).

Similarly, therapeutic strategies can use human NK cells, or CAR-NK cells according to the invention, with the aim of stimulating their activity by enhancing activation or proliferation with transgenes such as NKG2D, IL-12, IL-15, or IL-18.

In the context of CAR-T cells TCR transgenic T cells, NK cells or CAR-NK cells, the On/Off modulation enabled by the NUTRIREG system is also very important to allow both (i) to have the beneficial effect of the transiently expressed gene and (ii) to limit its potentially toxic effect if it is expressed for too long (example of cJUN, which may have an oncogenic effect).

Accordingly, in some embodiments, the cellular immunotherapy with the human CAR-T cells or TCR transgenic T cells, or human NK cells or CAR-NK cells of the invention is for treating cancer, in particular a solid cancer.

For cancer therapy, the skilled person will select in particular a human CAR-T cell, TCR transgenic T or CAR-NK cell that is specific for a tumor antigen of the subject having cancer.

In these aspects, the transgene stimulates efficacy of cellular immunotherapy with said human CAR-T cell, transgenic TCR T cell, NK cell or CAR-NK cell, or curbs toxicity induced by said CAR-T cell or transgenic TCR T cell. In particular, the transgene prevents CAR-T cell exhaustion or transgenic TCR T cell exhaustion.

In some other embodiments, the cellular immunotherapy with the human CAR-T cells or TCR transgenic T cells of the invention is for treating an auto-immune disease. In particular, for an auto-immune disease caused by the production of autoantibodies, the human CAR-T cells or TCR transgenic T cells will target auto-antibodies producing B cells. For an auto-immune disease caused by cytotoxic T cells (such as in colitis, multiple sclerosis, or type 1 diabetes), the human CAR-T cells or TCR transgenic T cells of the invention will target the pathogenic autoimmune T cells.

According to these aspects, the human T-cell is a chimeric antigen receptor T (CAR-T) cell or a T cell receptor (TCR) transgenic T cell. Depending on the therapeutic use, the CAR-T cell or TCR transgenic T cell is specific for an auto-antibody, or a pathogenic auto-immune T-cell. In these aspects, the transgene stimulates efficacy of cellular immunotherapy with said CAR-T cell or transgenic TCR T cell, or curbs toxicity induced by said CAR-T cell or transgenic TCR T cell. In particular, the transgene prevents CAR-T cell exhaustion or transgenic TCR T cell exhaustion.

In some embodiments, the cellular immunotherapy is for controlling allograft rejection of solid transplant.

Furthermore, cell therapy with ex vivo-expanded autologous Tregs is one of the most promising approaches to regulate alloimmunity and reduce immunosuppression. Preclinical studies have shown that the potency of Treg therapy can be markedly enhanced by the use of Tregs specific for donor alloantigens (i.e. donor-specific (ds) Tregs). The CAR-Treg technology was used to generate dsCAR-Tregs, and these cells were found to suppress humoral immunity, and delay allograft rejection in unsensitized, immunocompetent recipients (Sicard et al., Am J Transplant. 2020; 20:1562-1573). Accordingly, in some embodiments, the cellular immunotherapy is for controlling allograft rejection with human CAR-T cells or TCR transgenic T cells according to the invention that are specific for donor alloantigen(s). In some aspects, the transgene extends lifespan of said donor specific CAR-T cell or donor specific transgenic TCR T cell, and in particular prevents dsCAR-T cell exhaustion or transgenic dsTCR T cell exhaustion, as already described above.

### Method of Preparing NUTRIREG T Cell or NK Cell

It is provided a method of preparing a human T cell or NK cell according to the invention, wherein a human T cell or NK cell is transfected or transduced with a vector comprising a nucleic acid construct which comprises:
i) a regulatory polynucleotide comprising a minimal promoter and at least one AARE (amino acid response element) nucleic acid sequence, said regulatory polynucleotide being activated in a subject upon consumption of a diet deficient in at least one essential amino acid; and
ii) a transgene which is placed under the control of the said regulatory polynucleotide.

It is also provided a method of preparing a human T cell or NK cell according to the invention, wherein a human T cell or NK cell is transfected or transduced with a vector comprising a nucleic acid construct which comprises:
i) a regulatory polynucleotide comprising a minimal promoter and at least one AARE (amino acid response element) nucleic acid sequence, said regulatory polynucleotide being activated in said T or NK cell when said T or NK cell is activated, and upon exposure to a deficiency in at least one essential amino acid, for instance when cultivated in a medium deficient in at least one essential amino acid; and
ii) a transgene which is placed under the control of the said regulatory polynucleotide.

The vector be a synthetic vector (cationic lipids, polymer liposomes, etc.), a plasmid, or a viral vector.

If desired, it may be combined with one or more substances that improve the vector's transfection efficacy and/or stability. These substances are widely documented in the literature available to a person skilled in the art (see, e.g., Felgner et al., 1987, Proc. West. Pharmacol. Soc. 32, 115-121; Hodgson and Solaiman, 1996, Nature Biotechnology 14, 339-342; Remy et al., 1994, Bioconjugate Chemistry 5, 647-654). By way of non limiting illustration, they may be polymers, cationic lipids, liposomes, nuclear proteins, or neutral lipids. These substances may be used alone or in combination. One possible combination is a recombinant plasmid vector combined with cationic lipids (DOGS, DC-CHOL, spermine-chol, spermidine-chol, etc.) and neutral lipids (DOPE).

A wide selection of plasmids can be used in the context of the present invention. They may be cloning and/or expression vectors. In general, they are known in the art, and a number of them are commercially available, but it is also possible to construct or modify them using genetic manipulation techniques. By way of examples, we may mention the plasmids derived from pBR322 (Gibco BRL), pUC (Gibco BRL), pBluescript (Stratagene), pREP4, pCEP4 (Invitrogene), or p Poly (Lathe et al., 1987, Gene 57, 193-201). Preferably, a plasmid implemented in the present invention contains a replication origin that ensures the start of replication in a producer cell and/or a host cell (for example, the ColEl origin will be used for a plasmid to be produced in E. coli, and the oriP/EBNAI system will be used if it is to be self-replicative in a mammal host cell, Lupton and Levine, 1985, Mol. Cel. Biol. 5, 2533-2542; Yates et al., Nature 313, 812-815). It may also include a selection gene for selecting or identifying the transfected cells (complementation of an auxotrophic mutation, gene coding for resistance to an antibiotic, etc.). It may also include additional elements that improve its maintenance and/or its stability in a given cell.

When a viral vector is involved, it may be a vector derived from an adenovirus, a lentivirus, a retrovirus, an adenovirus-associated virus (AAV), a herpes virus, an alphavirus, a parvovirus, a poxvirus (fowlpox, canarypox, vaccinia viruses, in particular of the MVA (Modified Virus Ankara) or Copenhagen strains, etc.) or a foamy virus. Preferably, a non replicative and, optionally, non integrative vector is used. Retroviruses have the property of infecting and becoming predominantly integrated into dividing cells and are therefore especially well-suited to an application in anticancer therapy. A suitable retroviral vector for the implementation of the present invention comprises LTR (Long Terminal Repeat) terminal sequences and an encapsidation region. It may derive from a retrovirus of any origin (murine, primate, feline, human, etc.) and, in particular, may derive from a retrovirus selected from the group including MoMuLV (Moloney Murine Leukemia Virus), MVS (Murine

Sarcoma Virus), or Friend Murine Retrovirus (Fb29). It is propagated in an encapsidation line that is able to provide in trans the gag, pol, and/or env viral polypeptides needed for the constitution of a viral particle. These types of lines are described in the literature (PA317, Psi CRIP GP+Am-12, etc.). The retroviral vector of the invention may comprise modifications, in particular at the LTRs (replacement of the promoter region by a eukaryote promoter) or at the encapsidation region (replacement by a heterologous encapsidation region, e.g., of the VL30 type).

In a preferred embodiment of the invention, the vector is a lentiviral vector, an adenoviral vector, or a vector derived from an adenovirus-associated virus (AAV).

According to an advantageous embodiment, the viral vector used according to the invention may be in the form of a DNA vector or of an infectious viral particle.

The invention will be further illustrated by the following figures and examples.

### FIGURES

FIG. 1: General principle of NUTRIREG.

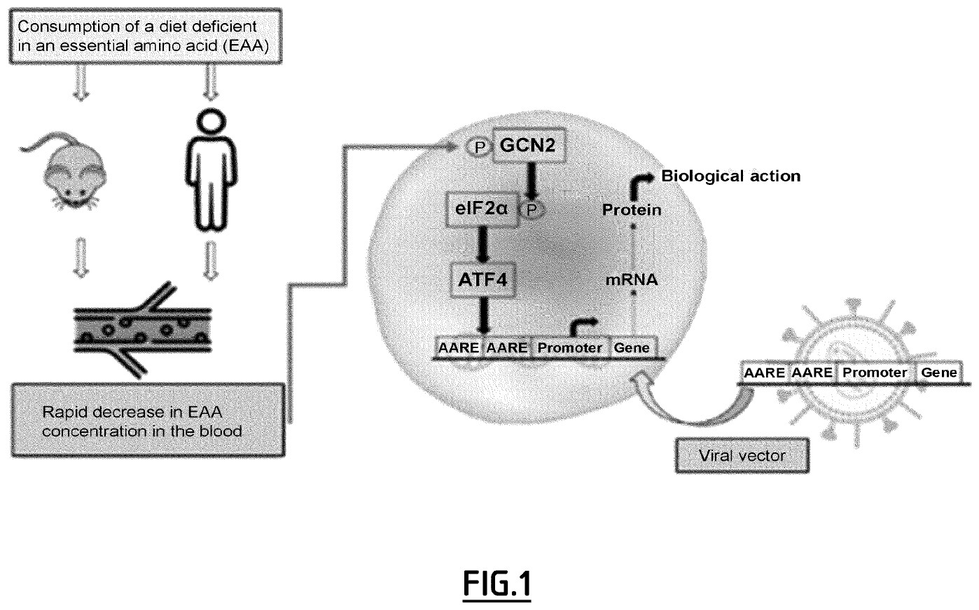

FIG. 2: Conditions of inducibility of a target gene (TRB3) on the GCN2-ATF4 pathway in human T cells following an EAA starvation.

FIG. 3: Induction of the expression of the luciferase transgene under the control of NUTRIREG. NT cells: non-transduced cells; LT AARE-TK-LUC: T cells transduced with the 2xAARE-TK-LUC construct.

FIG. 4: Induction of the expression of the GFP transgene under the control of NUTRIREG. NT cells: non-transduced cells; LT AARE-TK-eGFP: T cells transduced with the 2xAARE-TK-eGFP construct.

FIG. 5: Co-expression analyzes of T cells and eGFP protein activation markers in flow cytometry.

FIG. 6: Principle of the use of NUTRIREG to induce the expression of IL-10 in T cells.

FIG. 7: Induction of mRNA (A) and protein (B) expression of the IL-10 transgene under the control of NUTRIREG. NT cells: non-transduced cells; LT AARE-BG-IL10: T cells transduced with the 2xAARE-BG-IL10 construct.

FIG. 8: Reversibility of NUTRIREG-mediated induction of IL-10 transgene mRNA (A) and protein (B) expression.

FIG. 9: Effect of NUTRIREG-dependent IL-10 expression on mRNA expression of cytokines characteristics of type 1 regulatory T cells (Tr1).

FIG. 10: Schematic representation of experimental design. NXG immunodeficient mice received 10.10⁶ human T cells transduced with a viral vector containing AARE-TK-IL-10 construct and 5.10⁶ human irradiated PBMC by tail vein injection. Seven days later, and then once a week until week 3, mice were fed with a control diet or leucine-deficient diet for 16 hours, prior to blood sampling for flow cytometry analysis and IL-10 measurement.

FIG. 11: Monitoring the expansion of human CD45⁺ cells in mouse blood. PBMCs were analysed by flow cytometry every week from week 1 to week 3, after the mice had consumed the control diet or leucine-deficient diet for 16 h. Results are expressed as percentage of human CD45⁺ cells (left) or in percentage of CD4⁺ or CD8⁺ cells among human CD45⁺ cells (Student's t test—Leu (16 h) versus Ctl: ns non-significant; n=4-5 per condition; error bars, s.e.m.).

FIG. 12: Monitoring of human IL-10 in mouse plasma. Plasmatic human IL-10 concentration was assayed by ELISA according to Methods section, every week from week 1 to week 3, just after mice consumption of control diet or leucine-deficient diet for 16 h (n=3-4 per condition; error bars, s.e.m.; on right: linear regression analysis).

FIG. 13: NUTRIREG-T cells application with the FoxP3 therapeutic gene. (A) Analysis of FoxP3 mRNA level by RT-qPCR in non-transduced T cells (NT cells) and in T cells transduced with the AARE-TK-FoxP3 construct (LT) (MOI 10), cultivated in a control medium (Ctl) or deficient in Leucine during 16 h (−Leu (16 h)). Expression normalised on actine expression. ANOVA test (−leu vs ctl et NT cells vs LT AARE-TK-FoxP3), ns not significant, ***p<0.001, n=3/condition. (B) Analysis of FoxP3 protein level, eIF2α protein level and phosphorylated eIF2α protein level by Western Blot in non-transduced T cells (NT cells) and in T cells transduced with the AARE-TK-FoxP3 construct (LT) (MOI 10), cultivated in a control medium (Ctl) or deficient in Leucine during 16 h (−Leu (16 h)).

FIG. 14: Analysis of TGF-β mRNA level by RT-qPCR in non-transduced T cells (NT cells) and in T cells transduced with the AARE-TK-FoxP3 construct (LT) (MOI 10), cultivated in a control medium (Ctl) or deficient in Leucine during 16 h (−Leu (16 h)). Expression normalised on actine expression. ANOVA test (−leu vs ctl et NT cells vs LT AARE-TK-FoxP3), ns not significant, *p<0.05, n=3/condition.

### EXAMPLES

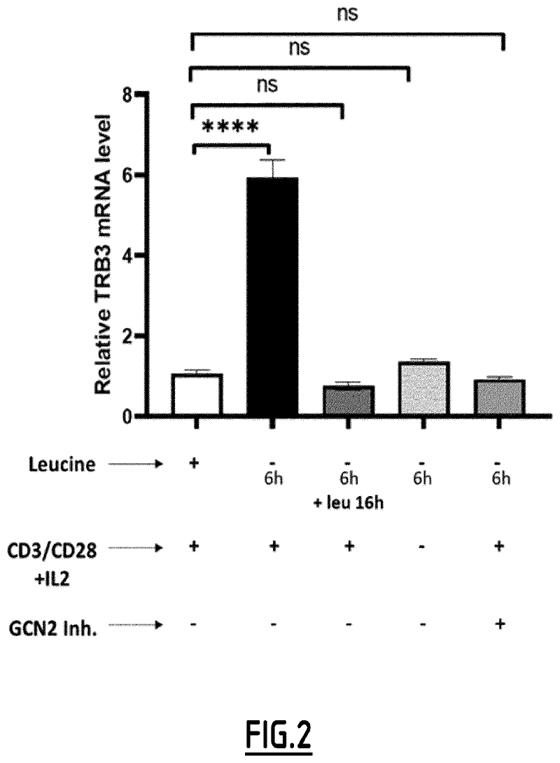

### Example 1: Validation of GCN2-ATF4 Pathway Inducibility in Human T Cells Following an EAA Starvation (FIG. 2)

In order to subsequently be able to use NUTRIREG in human T cells, we first validated that the GCN2-ATF4 pathway is inducible by an EAA starvation in this cell type. Data from the literature previously highlighted the activation of this signaling pathway in mice T cells in response to IDO (Indoleamine 2.3-Dioxygenase), halofuginone, or asparaginase (Munn et al., Immunity 2005, Vol. 22, 633-642; Van de Velde et al., Cell reports 17.9 (2016): 2247-2258; Sundrud et al., Science 324.5932 (2009): 1334-1338.; Bunpo et al., Journal of Nutrition 2010, 140, 2020-2027), but only a few data was available on GCN2-ATF4 pathway activation in response to AA deficiency in human T cells.

We have thus carried out in vitro experiments with human T cells purified from “buffy coat” from the French Blood Establishment, intended for research. We cultured these cells in control medium or in leucine-deficient medium.

We demonstrated that (FIG. 2):
(1) The expression of a known target gene (TRB3) of the GCN2-ATF4 pathway can be significantly induced by a 6 h EAA starvation (leucine or any other EAA) in T cells.
(2) The induction of the GCN2-ATF4 pathway by a short-time leucine starvation is rapidly reversible in T cells, from 16 hours after the addition of leucine in the starved medium.
(3) Importantly, the activation of T cells (by anti-CD3 and CD28 antibodies, in the presence of IL-2) is essential for the induction of the GCN2-ATF4 pathway in response to EAA starvation. The fact that the activation of the T lymphocyte is essential for the activation of GCN2 kinase, had been described in mice T cells following culture in a tryptophan-deficient medium. (Munn et al., Immunity 2005, Vol. 22, 633-642; Van de Velde et al., Cell reports 17.9 (2016): 2247-2258), but no information was available for human T lymphocytes.
(4) The induction of the expression of TRB3, a target gene of the GCN2-ATF4 pathway, in response to a short-time leucine starvation is fully dependent on the GCN2 kinase; this was demonstrated via the use of a pharmacological inhibitor of GCN2.

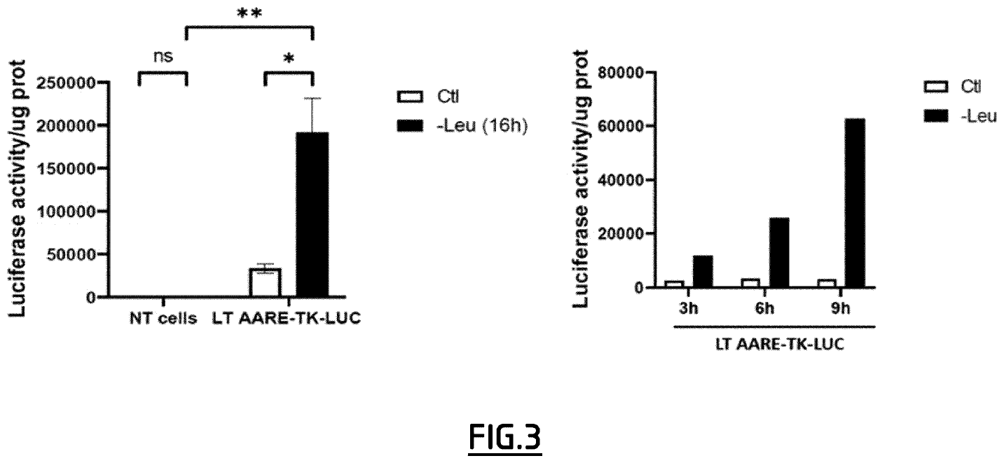

### Example 2: Validation of NUTRIREG Functionality in Human T Cells with the Luciferase Reporter Gene (FIG. 3)

We then assessed the functionality of NUTRIREG in human T cells using two reporter genes. The first reporter gene used was the luciferase gene, inserted into a 2xAARE-TK-LUC construct and transduced through a lentiviral vector. Transduction was performed 24 hours after activation of T cells by magnetic beads loaded with anti-CD2/CD3/CD28 antibodies, in the presence of IL-2. The cells were then deprived of leucine 8 days after their activation, followed by a measurement of the luciferase activity.

We thus demonstrated that a 16 h leucine starvation does indeed induce the expression of the luciferase transgene via NUTRIREG in T cells, in comparison to (i) the transduced T cells cultured in a control medium, and (ii) the non-transduced T cells deprived for leucine for 16 hours. This induction is detectable from 3 hours of leucine deficiency with an increase in the expression of the transgene when the duration of the EAA deficiency increases (6 h, 9 h) (FIG. 3).

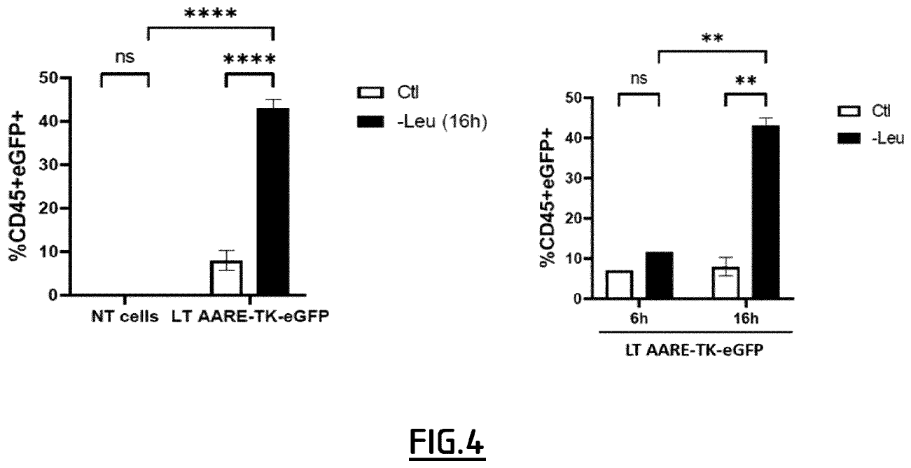

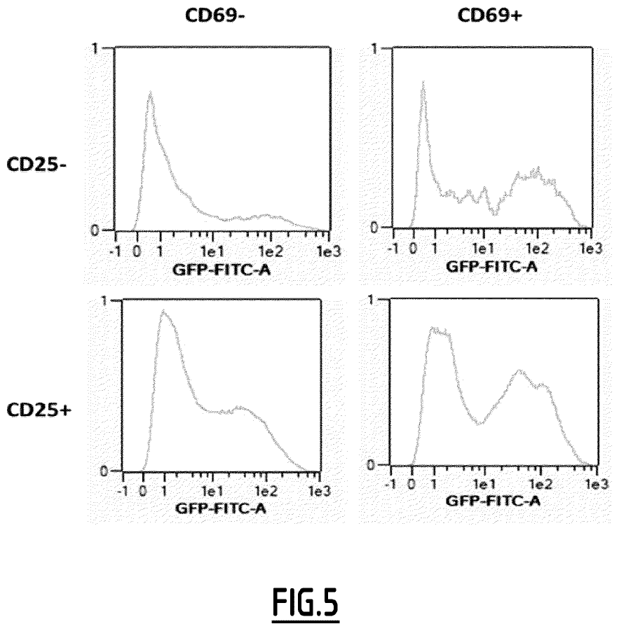

### Example 3: Validation of NUTRIREG Functionality in Human T Cells In Vitro with the eGFP Reporter Gene (FIGS. 4-5)

The second reporter gene evaluated was the eGFP gene, inserted into a 2xAARE-TK-eGFP construct and transduced through a lentiviral vector. Transduction was performed 24 hours after activation of T lymphocytes by magnetic beads loaded with anti-CD2/CD3/CD28 antibodies, in the presence of IL-2. The cells were then deprived of leucine 8 days after their activation, with measurement of the fluorescence emitted by the eGFP protein by flow cytometry.

We demonstrated that a 16 h leucine starvation does indeed induce the expression of the eGFP transgene via NUTRIREG in T cells, in comparison to (i) the transduced T cells cultured in a control medium, and (ii) the non-transduced T cells deprived for leucine for 16 hours. This induction is not possible on shorter leucine starved times, probably due to a problem of stability of the eGFP protein (FIG. 4).

In addition, analyzes of the co-expression of T cells activation markers (CD69 as early activation marker and CD25 as later activation marker) and of the eGFP protein in flow cytometry enabled us to confirm the key role of T cells activation to induce the expression of the transgene under the control of NUTRIREG. Indeed, the non-activated cells (CD25⁻ CD69⁻) do not express eGFP, those moderately activated (CD69⁻ CD25⁺ or CD69⁺ CD25⁻) express it weakly, and the strongly activated cells (CD69⁺ CD25⁺) are those which express the most eGFP protein (FIG. 5).

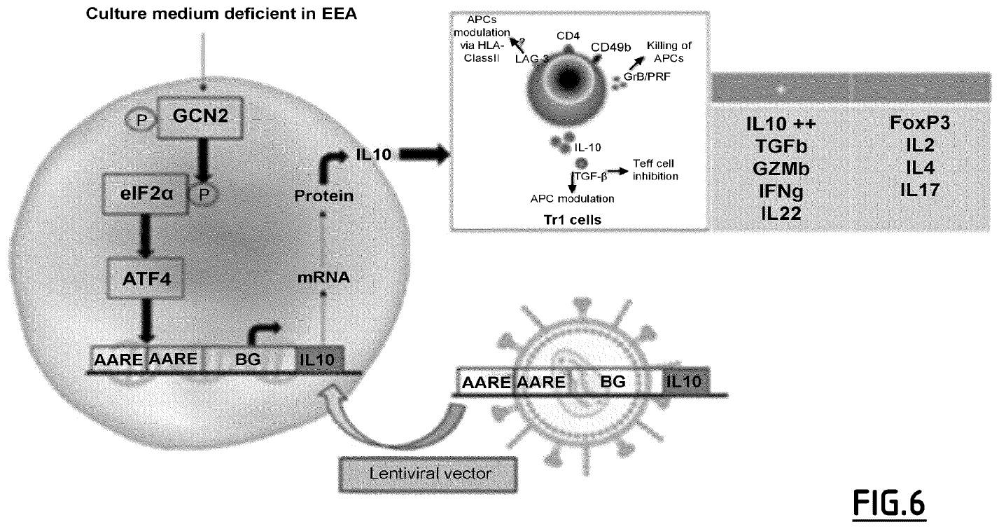

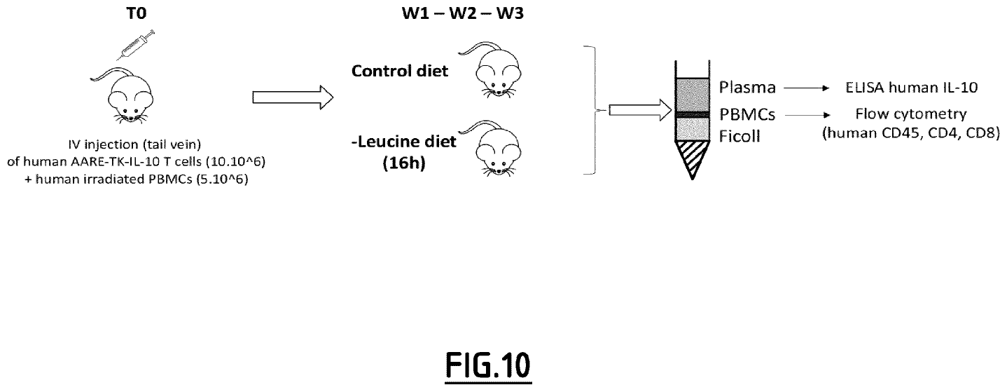

### Example 4: NUTRIREG-T Cells Application with the Interleukin-10 (IL-10) Therapeutic Gene (FIGS. 6-9) and Results In Vivo (FIGS. 10-12)

With the aim of using the NUTRIREG technology for therapeutic purposes in a model of treatment or prevention of GvHD (Graft versus Host disease), we designed a 2xAARE-BG-IL10 construct in which is inserted the Interleukin-10 (IL-10) cDNA placed downstream the minimum Beta-globin (BG) promoter and the 2XAARE sequence (FIG. 6). Indeed, it has been demonstrated in the literature that the transduction of T cells with an IL-10 lentivirus allows to (i) direct the immune response towards a Tr1 type anti-inflammatory response (T regulatory type 1) and (ii) prevent the appearance of GvHD in mouse models (Andolfi et al., Molecular Therapy 2012, vol. 20, 1778-1790; Locafaro et al., Molecular Therapy 25.10 (2017): 2254-2269).

There are also early phase human clinical trials aimed at directing the immune response towards a Tr1 phenotype for the purpose of preventing GvHD. To this end, donor T cells are made anergic towards host cells by culturing these donor T cells with host CD3-depleted mononuclear cells (PBMC), in the presence of IL-10. Anergic T cells are then reinjected to the recipient a few weeks after the allogeneic hematopoietic stem cell transplant (Bacchetta et al., Frontiers in immunology 5 (2014): 16.). These type 1 regulatory T cells (Tr1) have a specific cytokine profile since they strongly express IL-10, and to a lesser extent TGFβ, GZMb, IFNγ and IL-22. On the other hand, they do not express IL-2, IL4 and IL17, nor the FoxP3 transcription factor (Gregori et al., Frontiers in immunology 6 (2015): 593.).

In our experiments, transduction with the lentivirus carrying the 2xAARE-BG-IL-10 construct was carried out 24 hours after the activation of the T cells by magnetic beads loaded with anti-CD2/CD3/CD28 antibodies, in the presence of IL-2. The cells were then deprived of leucine 8 days after their activation, with measurement of the expression of (i) the IL-10 mRNA by RT-qPCR, (ii) the IL-10 protein by ELISA or flow cytometry, and (iii) the different cytokines of the Tr1 phenotype by RT-qPCR (FIG. 6).

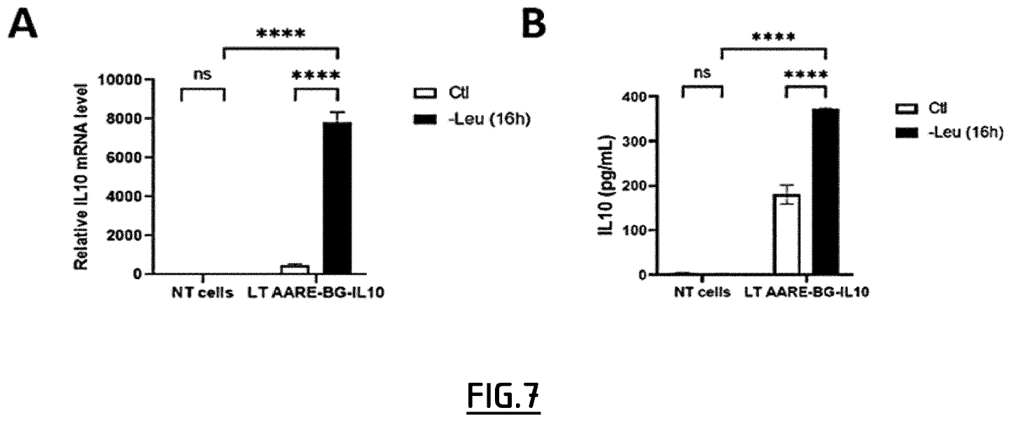

We have thus demonstrated that a 16 h leucine starvation does indeed induce the mRNA and protein expression of the IL-10 transgene via NUTRIREG, in comparison to (i) the transduced T cells cultured in control medium, and (ii) the non-transduced T cells starved for leucine (FIG. 7A-B).

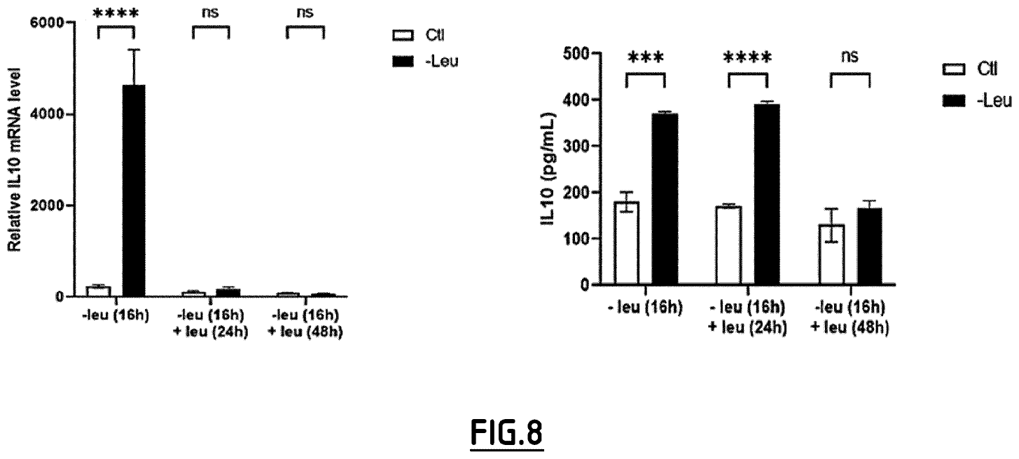

We also checked that the addition of leucine in the culture medium following the starvation indeed stop the transgene expression in the transduced T cells, at 24 h for the expression of mRNA and at 48 h for the expression of the IL-10 protein (FIG. 8A-B).

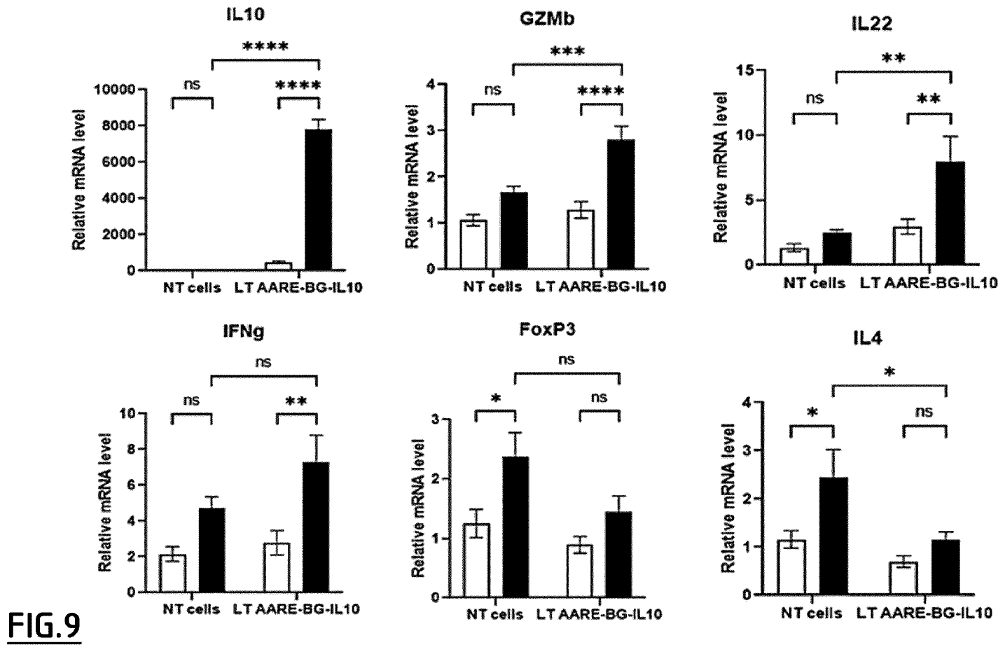

Finally, we were able to show that a transient expression of the IL-10 transgene via NUTRIREG allows to direct the immune response towards a Tr1 type response as shown by the cytokine and transcription factors profile (mRNA): IL-10⁺⁺ GZMb⁺ IL22⁺ IFNγ⁺ FoxP3⁻ IL4⁻ (FIG. 9).

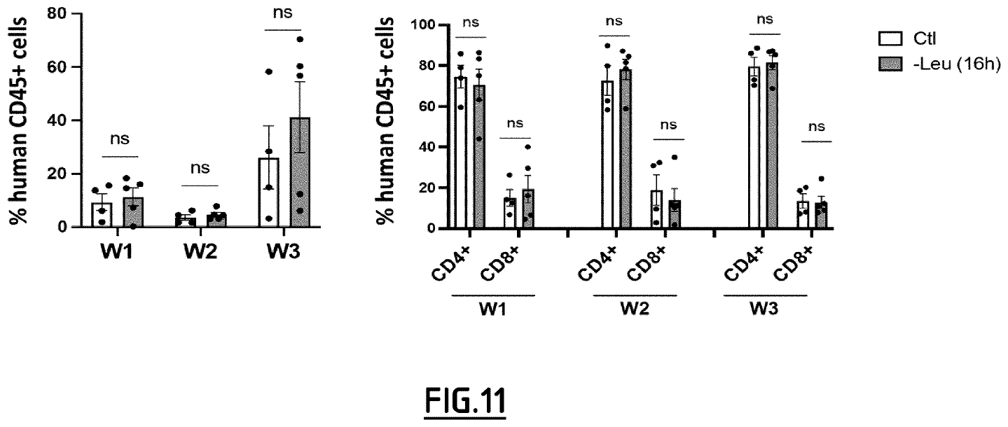

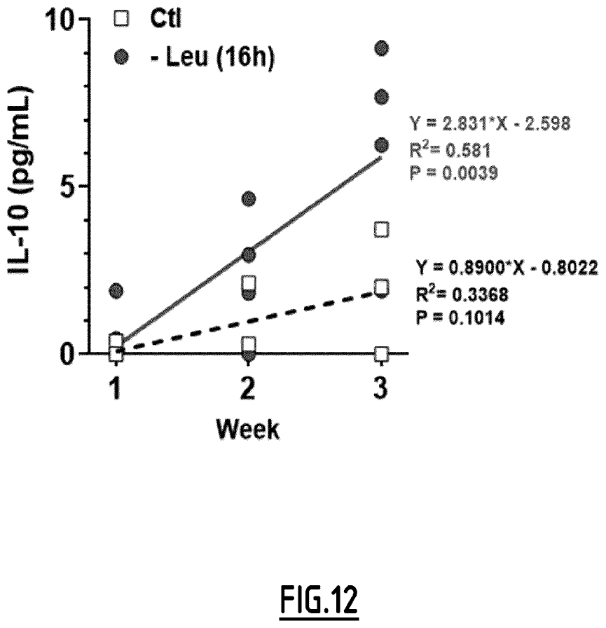

We lastly investigated the capacity of a diet deficient in leucine to upregulate the AARE-driven IL-10 expression in human T cells injected into mice. Indeed, the lack of any of the EAAs in the diet of mammals represents a potential inducer of the AARE-transgene expression. As activation of T cells is mandatory for the EAA-induction of the GCN2-ATF4 pathway, we set up a mouse model in which we injected transduced human T cells in association with autologous irradiated human PBMCs. We assumed that transduced human T cells would be transiently activated by irradiated PBMCs and that this activation would be sufficient for the GCN2-ATF4 pathway to be inducible by an EAA nutritional deficiency. Briefly, immunodeficient NXG mice were injected into the tail vein with (i) human T cells transduced with lentiviruses carrying the 2xAARE-TK-IL-10 construct and (ii) human irradiated PBMCs, and were then fed for 16 h, once a week until week 3, with either a control diet or a leucine-devoid diet (FIG. 10). Weekly flow cytometry analysis of PBMCs enabled us to follow the correct expansion of human CD45⁺ cells, with no significant impact of leucine-deficient diet on cells expansion nor on CD4⁺ or CD8⁺ phenotype (FIG. 11). Human IL-10 increased linearly with time in the leucine-deficient group (R²=0.581; P=0.004) and was not significantly changed over time in control group (FIG. 12).

Collectively, these results demonstrate that the NUTRIREG technology is able to regulate, in vitro and in vivo, a therapeutic gene expression, such as IL-10, in activated human T cells.

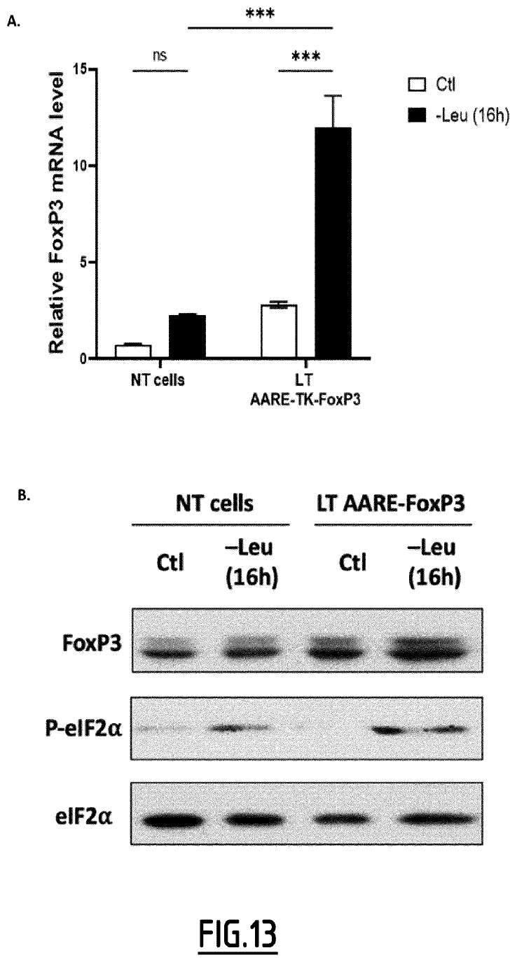

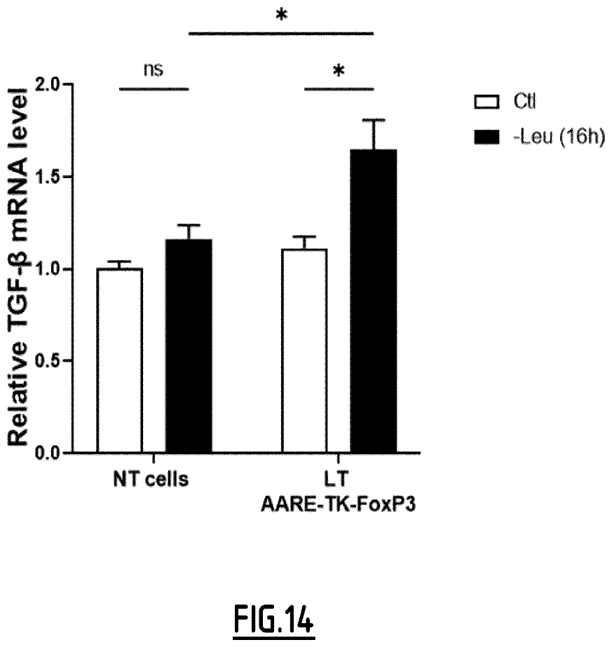

### Example 5: NUTRIREG-T Cells Application with the FoxP3 Transcription Factor (FIGS. 13-14)

Several publications in the literature demonstrate that transduction of T lymphocytes with the FoxP3 gene makes it possible to orient the phenotype of these T lymphocytes into the Treg FoxP3+ phenotype. For example, the injection of CD4⁺CD25⁻ T cells (with no constitutive expression of FoxP3), transduced with a retrovirus carrying FoxP3, into immunodeficient RAG-/-mice, allows to direct these cells towards a phenotype CD4⁺CD25⁺, to suppress the cytotoxic CD8+ reaction, and to prevent the appearance of inflammatory colitis. These results in mice were confirmed in vitro in human cells by lentiviral transduction of FoxP3. In this context, we worked on the overexpression via NUTRIREG of the transcription factor Foxp3, with the aim of transiently and reversibly polarizing T lymphocytes into the Treg phenotype. We thus designed a 2xAARE-TK-FoxP3 (human) transgene which we transduced into activated human T lymphocytes (activated for 24 hours). Eight days after transduction, we transferred our cells in a control medium or a leucine-deficient medium for 16 hours. We have thus observed a transduction of FoxP3 under the control of NUTRIREG makes it possible to induce the expression of this transgene following a culture in leucine-deficient medium for 16 hours. This expression is induced at the transcriptional level (FIG. 13A) as well as at the translational level (FIG. 13B). This expression of FoxP3 is associated with increased expression of the anti-inflammatory cytokine TGF-β (FIG. 14), as well as the cytotoxic molecules Granzyme B and Perforin 1, which are among the characteristics exhibited by FoxP3⁺ Tregs.

1. A human T cell or NK cell comprising a nucleic acid construct which comprises:
i) a regulatory polynucleotide comprising a minimal promoter and at least one AARE (amino acid response element) nucleic acid sequence, said regulatory polynucleotide being activated in said T or NK cell when said T or NK cell is activated, and upon exposure to a deficiency in at least one essential amino acid; and
ii) a transgene which is placed under the control of the said regulatory polynucleotide,
wherein the transgene will be expressed only in activated T or NK cells.

2. The human T cell or NK cell according to claim 1, which is a human T cell and wherein the human T-cell is a chimeric antigen receptor T (CAR-T) cell or a T cell receptor (TCR) transgenic T cell.

3. The T cell or NK cell according to claim 1, which is a human NK cell or a human CAR-NK cell.

4. The human T cell or NK cell according to claim 1, wherein the human T cell or NK cell is select from a group consisting of CAR-T cell, TCR transgenic T cell, human NK cell, and human CAR-NK cell,
and wherein the transgene stimulates efficacy of cellular immunotherapy with said CAR-T cell, transgenic TCR T cell, human NK cell or human CAR-NK cell, or the transgene curbs toxicity induced by said CAR-T cell or transgenic TCR T cell.

5. The human T cell or NK cell according to claim 1, which is a human T cell, and wherein the human T cell is a human CAR-T cell or transgenic TCR T cell and wherein the transgene reverses or delays CAR-T cell exhaustion or transgenic TCR T cell exhaustion, or encodes an inhibitory receptor or immunosuppressive factor.

6. The human T cell or NK cell according to claim 1, which is a human T-cell intended for allogeneic transplantation or for controlling allograft rejection of solid transplant.

7. The human T cell according to claim 1, which is a human T-cell intended for allogeneic transplantation or for controlling allograft rejection of solid transplant, and wherein the transgene promotes human T cell differentiation into regulatory T cells (Treg or Tr1).

8. The human T cell according to claim 1, which is a human T-cell intended for allogeneic transplantation or for controlling allograft rejection of solid transplant, and wherein the human T-cell is intended for allogeneic transplantation and the transgene stimulates efficacy of cellular immunotherapy with said human T-cell.

9. The human T cell or NK cell according to any one of claim 1, wherein:
a) the amino acid response element (AARE) nucleic acid sequence is selected in a group consisting of sequences SEQ ID NO: 1, SEQ ID NO: 2, SEQ ID NO: 3, SEQ ID NO: 4 and SEQ ID NO: 5; and/or
b) the regulatory polynucleotide comprises at least two AARE nucleic acid sequences.

10. A method of treatment by cellular immunotherapy in a subject in need thereof comprising the human T cell or NK cell according to claim 1. wherein transgene expression will be only induced in the T or NK cells once they are activated in the subject.

11. The method of treatment by cellular immunotherapy according to claim 10, wherein the cellular immunotherapy is for treating cancer, for controlling allograft rejection of solid transplant, or for treating an auto-immune disease.

12. The method of treatment by cellular immunotherapy according to claim 10, wherein the cellular immunotherapy is for treating cancer.

13. The method of treatment by cellular immunotherapy according to claim 10,
wherein the cellular immunotherapy is for treatment of a hematologic cancer by allogeneic transplantation comprising the human T cell, or wherein the cellular immunotherapy is for controlling allograft rejection of solid transplant comprising the human T cell,
and wherein the human T cell a human CAR-T cell or transgenic TCR T cell and wherein the transgene reverses or delays CAR-T cell exhaustion or transgenic TCR T cell exhaustion, or encodes an inhibitory receptor or immunosuppressive factor.

14. The method for treatment by cellular immunotherapy according to claim 10, wherein the cellular immunotherapy is for treatment of a hematologic cancer by allogeneic transplantation comprising the human T cell, and wherein:
the expression of the transgene promotes human T cell differentiation into Treg or Tr1 and prevents or treats graft versus host disease (GvHD), or
the expression of the transgene stimulates efficacy of cellular immunotherapy with said human T-cell inducing or stimulating graft versus leukemia (GvL) effect.

15. The method for treatment by cellular immunotherapy according to claim 10, wherein the cellular immunotherapy is for controlling allograft rejection of solid transplant comprising a human CAR Treg, or wherein the cellular immunotherapy is for treating an auto-immune disease.

16. The method for treatment by cellular immunotherapy according to claim 10, wherein the subject administered with the T cell or NK cell further receives a diet deficient in at least one essential amino acid to induce transgene expression in human T cells or NK cells that are activated.

17. A method of preparing a human T cell or NK cell as defined in claim 1, wherein a human T cell or NK cell is transfected or transduced with a vector comprising a nucleic acid construct which comprises:
i) a regulatory polynucleotide comprising a minimal promoter and at least one AARE (amino acid response element) nucleic acid sequence, said regulatory polynucleotide being activated in said T or NK cell when said T or NK cell is activated, and upon exposure to a deficiency in at least one essential amino acid; and
ii) a transgene which is placed under the control of the said regulatory polynucleotide.

18. The human T cell or NK cell according to claim 1, wherein the human T is human CAR-T cell, or transgenic TCR T cell,
and wherein the transgene reverses or delays CAR-T cell exhaustion or transgenic TCR T cell exhaustion, or encodes an inhibitory receptor or immunosuppressive factor,
and wherein the transgene stimulates efficacy of cellular immunotherapy with said CAR-T cell, or said transgenic TCR T cell, or the transgene curbs toxicity induced by said CAR-T cell or transgenic TCR T cell.

19. The method of treatment by cellular immunotherapy according to claim 10,
wherein the cellular immunotherapy is for treatment of a hematologic cancer by allogeneic transplantation comprising the human T cell, or wherein the cellular immunotherapy is for controlling allograft rejection of solid transplant comprising the human T cell,
and wherein the human T is human CAR-T cell, or transgenic TCR T cell, and wherein the transgene reverses or delays CAR-T cell exhaustion or transgenic TCR T cell exhaustion, or encodes an inhibitory receptor or immunosuppressive factor,
and wherein the transgene stimulates efficacy of cellular immunotherapy with said CAR-T cell, or said transgenic TCR T cell, or the transgene curbs toxicity induced by said CAR-T cell or transgenic TCR T cell.


SEQUENCE LISTING (6 sequences)

SEQ ID NO: 1 - DNA, 16 bp, synthetic construct
  source 1..16: mol_type = other DNA
cggtttgcatcacccg

SEQ ID NO: 2 - DNA, 16 bp, synthetic construct
  source 1..16: mol_type = other DNA
aacattgcatcatccc

SEQ ID NO: 3 - DNA, 16 bp, synthetic construct
  source 1..16: mol_type = other DNA
gaagtttcatcatgcc

SEQ ID NO: 4 - DNA, 16 bp, synthetic construct
  source 1..16: mol_type = other DNA
agcgttgcatcacccc

SEQ ID NO: 5 - DNA, 16 bp, synthetic construct
  source 1..16: mol_type = other DNA
gatattgcatcagttt

SEQ ID NO: 6 - DNA, 317 bp, synthetic construct
  source 1..317: mol_type = other DNA
ggtaccgattagctccggtttgcatcacccggaccgggggattagctccggtttgcatcacccggaccgggggattagctccggtttgcatcacccggaccgggggccgggcgcgtgctagcgattagctccggtttgcatcacccggaccgggggattagctccggtttgcatcacccggaccgggggattagctccggtttgcatcacccggaccggggactcgaggtccacttcgcatattaaggtgacgcgtgtggcctcgaacaccgagcgaccctgcagcgacccgcttaacagcgtcaacagcgtgccgc